(19) United States
(12) Patent Application Publication
(10) Pub. No.: US 2024/0390308 A1
(43) Pub. Date: Nov. 28, 2024

(54) APPLICATION METHOD OF POTASSIUM ION CHANNEL BLOCKER IN PREPARATION OF DRUGS FOR TREATING LIVER FIBROSIS

(71) Applicant: Affiliated Hospital of Zunyi Medical University, Zunyi (CN)

(72) Inventors: Rui Xie, Zunyi (CN); Jingyu Xu, Zunyi (CN); Qian Du, Zunyi (CN); Yanxia Hu, Zunyi (CN); Jianhong Ding, Zunyi (CN); Xianli Wu, Zunyi (CN); Xiaoxu Yang, Zunyi (CN); Jun Lou, Zunyi (CN); Qi Liu, Zunyi (CN); Zhuo Li, Zunyi (CN)

(21) Appl. No.: 18/348,332

(22) Filed: Jul. 6, 2023

(30) Foreign Application Priority Data
May 22, 2023 (CN) 2023105795396

(51) Int. Cl. A61K 31/18; A61P 1/16

(52) U.S. Cl. CPC A61K 31/18; A61P 1/16

(57) ABSTRACT

An application of potassium ion (K⁺) channel blocker in preparation of drugs for treating liver fibrosis is provided, which relates to the field of medical technologies. The K⁺ channel blocker is sotalol, the sotalol is originally used as an antiarrhythmic drug in clinic and has a property of inhibiting K⁺ channels. Experiments show that sotalol can effectively inhibit liver fibrosis and reduce expression of fibrosis-related protein alpha-smooth muscle actin (α-SMA), specifically by regulating expression and/or function of autophagy and epithelial-mesenchymal transition (EMT) molecules to achieve inhibition of hepatic stellate cell (HSC) fibrosis, so as to provide a new drug choice for clinical treatment of the liver fibrosis.

### TECHNICAL FIELD

The disclosure relates to the field of medical technologies, and more particularly to an application of potassium ion (K⁺) channel blocker in preparation of drugs for treating liver fibrosis.

### BACKGROUND

Liver fibrosis is a response to long-term effects caused by chronic damage to liver, with many pathogenic factors for the liver fibrosis, and further development can lead to cirrhosis and even liver cancer, which seriously threatens human health and life. Liver fibrosis stage is not only a common pathological basis for all chronic liver diseases, but also a key link for further development towards cirrhosis and hepatocellular carcinoma (HCC). In recent years, relevant studies have found that liver fibrosis, even early cirrhosis, can be reversed. Currently, how to effectively fight liver fibrosis is a research focus of domestic and foreign scholars.

Clinical treatment for anti-fibrosis mainly focuses on eliminating the causes of the liver fibrosis, includes the following measures: antiviral therapy, alcohol abstinence, diet control and the like, however, due to the disadvantages of long treatment courses, poor efficacy, multiple adverse reactions, poor patient compliance and the like, the prognosis of patients is poor. Therefore, finding new potential targets for anti-liver fibrosis to guide the clinical treatment of the liver fibrosis more precisely and effectively has positive significance for improving patients' prognosis. However, costs of developing new drugs are relatively high, it is may be considered that whether some traditional non-antifibrotic drugs can be found in clinical for treatment of the liver fibrosis.

Ion channels are a research hotspot in recent years. The ion channels are an extremely important class of proteins that regulates cell signals by strictly controlling the inflow or outflow of ions in cells or organelle and the ion channels, thereby affecting a wide range of physiological and pathological processes. The effect of the ion channels on the liver fibrosis has attracted increasing attention in the research of the ion channels and digestive system. Potassium channels have been proven to promote fibroblast function in various fibrotic diseases. In the liver, the potassium channels promote the liver fibrosis by promoting expression of fibrotic tissue and transforming growth factor beta (TGF-β) and activating hepatic stellate cells (HSCs). Because of the above association of the potassium channels with the liver fibrosis, it is expected to find a potassium blocker for the treatment of the liver fibrosis in clinic in response to the trend of drug reuse. Sotalol is a competitive β-adrenergic receptor antagonist, in addition to its class II (β-blocker) effects, sotalol has class III antiarrhythmic activity, which can increase action potential duration (APD) and prolong atrial and ventricular repolarization. In this situation, sotalol is also a potassium blocker that can inhibit expression of potassium channels. At present, research on sotalol is limited to its role in arrhythmia, and there are few reports on the effect and mechanism of sotalol on the liver fibrosis.

### SUMMARY

In view of the shortcomings of related art, the disclosure provides an application of a potassium ion (K⁺) channel blocker in preparation of drugs for treating liver fibrosis.

To achieve the above objectives, the disclosure is implemented through the following technical solutions.

The disclosure provides an application of a K⁺ channel blocker in preparation of drugs for treating liver fibrosis.

In an embodiment, the K⁺ channel blocker is sotalol.

The disclosure has beneficial effects as follows.
1. Sotalol is originally used as an antiarrhythmic drug in clinic and has a property of inhibiting K⁺ channels. Experiments show that sotalol can inhibit liver fibrosis effectively and reduce expression of fibrosis-related protein alpha-smooth muscle actin (a-SMA), specifically by regulating expression and/or function of autophagy and epithelial-mesenchymal transition (EMT) molecules to achieve inhibition of hepatic stellate cell (HSC) fibrosis, so as to provide a new drug choice for clinical treatment of the liver fibrosis.
2. In the progress of research, sotalol is discovered that it can inhibit activated HSCs to improve degree of the liver fibrosis, specifically by inhibiting roles of the EMT and pathway of the autophagy.
3. The disclosure uses a K⁺ channel blocker (i.e., sotalol) to explore its effects on liver fibrosis sotalol for the first time, and sotalol can provide more choices for inhibiting activation of HSCs and improving even reversing the liver fibrosis.

### BRIEF DESCRIPTION OF DRAWINGS

FIGS. 1A-1D illustrate degrees of liver tissue fibrosis observed by using hematoxylin-eosin (HE) staining and Sirius-red staining methods according to an embodiment 1.

FIGS. 2A-2B illustrate statistical diagrams showing changes in levels of alanine transaminase (ALT) and aspartate transaminase (AST) in serum detected to the embodiment 1.

FIGS. 3A-3B illustrate changes in expression of alpha-smooth muscle actin (α-SMA) in liver tissue detected by immunohistochemistry according to an embodiment 2.

FIGS. 4A-4B illustrate changes in expression of α-SMA in the liver tissue detected by Western-blot according to an embodiment 3.

FIGS. 5A-5D illustrate changes in expression of autophagy-related index light chain 3 (LC3), protein 62 (p62) in the liver tissue detected by Western-blot according to the embodiment 3.

FIGS. 6A-6D illustrate changes in expression of epithelial-mesenchymal transition (EMT) mesenchymal-like cell markers N-cadherin and vimentin in the liver tissue detected by Western-blot according to the embodiment 3.

FIGS. 7A-7B illustrate changes in expression of an EMT mesenchymal-like cell marker zonula occludens-1 (ZO-1) in the liver tissue detected by Western-blot according to the embodiment 3.

FIG. 8 illustrates a statistical diagram of effects of sotalol and transforming growth factor beta 1 (TGF-β1) co-culture on proliferation of hepatic stellate cells (HSCs) detected by Cell Counting Kit-8 (CCK-8) according to an embodiment 4.

FIGS. 9A-9B illustrate changes in protein expression levels of α-SMA in HSCs detected by Western-blot to an embodiment 5.

FIGS. 10A-10B illustrate changes in protein expression levels of LC3 in HSCs detected

by Western-blot according to the embodiment 5.

FIGS. 11A-11B illustrate changes in protein expression levels of p62 in HSCs detected by Western-blot according to the embodiment 5.

FIGS. 12A-12B illustrate changes in protein expression levels of ZO-1 in HSCs detected by Western-blot according to the embodiment 5.

FIGS. 13A-13B illustrate changes in protein expression levels of N-cadherin in HSCs detected by Western-blot according to the embodiment 5.

FIGS. 14A-14B illustrate changes in protein expression levels of vimentin in HSCs detected by Western-blot according to the embodiment 5.

### DETAILED DESCRIPTION OF EMBODIMENTS

Technical solutions in embodiments of the disclosure will be clearly and completely described below in conjunction with the accompanying drawings. Apparently, the described embodiments are merely a part of the embodiments of the disclosure, not all of them. Based on the embodiments in the disclosure, all other embodiments obtained by those skilled in the art without creative work fall within the scope of protection of the disclosure.

Unless otherwise specified, the technical means used in the embodiments of the disclosure are conventional means known to those skilled in the art.

The disclosure provides an application of a potassium ion (K⁺) channel blocker in preparation of drugs for treating liver fibrosis. The K⁺ channel blocker is represented to improve liver fibrosis, specifically, the K⁺ channel blocker is sotalol.

The disclosure is further described below with reference to specific embodiments.

### Embodiment 1 Carbon Tetrachloride (CCl4)-induced Liver Fibrosis In Mice

Experimental materials include 60 healthy male C57 mice (20-25g/mouse), gavage needles, 1 milliliter (mL) syringes, CCl₄ solution, olive oil and sotalol solution.

Experimental method is as follows. The CCl₄ solution is dissolved in the olive oil solution (CCl₄: olive oil=1:4, V/V), prepared and used immediately, concentration is 0.1milliliters per 20 grams (0.1 mL/20 g), intragastrically administered twice a week for a total of 8 weeks to the mice. The sotalol solution is mixed with physiological saline, prepared and used immediately, concentration is 1 millimole per liter (mM), 0.2 mL/20 g, and intraperitoneally injected once a day for a total of 6-8 weeks to the mice. The mice are randomly divided into four groups: a. a normal control group; b. a model group, mice are intragastrically administered the CCl₄ for 8 weeks; c. a CCl₄ and sotalol group, mice are treated with the CCl₄ alone for 2 weeks, followed by concurrent administration of the sotalol and CCl₄ for 6 weeks; d. a sotalol group, mice are intraperitoneally injected the sotalol for 8 weeks. The mice are sacrificed in sequence at the 2^{nd}, 4^{th}, 6^{th}, and 8^{th} weeks of modeling, one for each group, liver samples of the mice are taken out and placed in a −80 Celsius degree (° C.) refrigerator for quick-frozen storage, and other livers are fixed in 4% paraformaldehyde for 24 hours and then embedded in paraffins. Severity of the liver fibrosis is observed through appearance, Sirius-red staining, and hematoxylin-eosin (HE) staining, if staining results indicate liver fibrosis, it is considered that the model is built successfully. Then, all mice are sacrificed, and eyeball blood of mice is taken to measure liver function indexes (alanine transaminase abbreviated as ALT, aspartate transaminase abbreviated as AST). In addition, the liver tissue is fixed with formaldehyde and embedded in paraffins for later use.

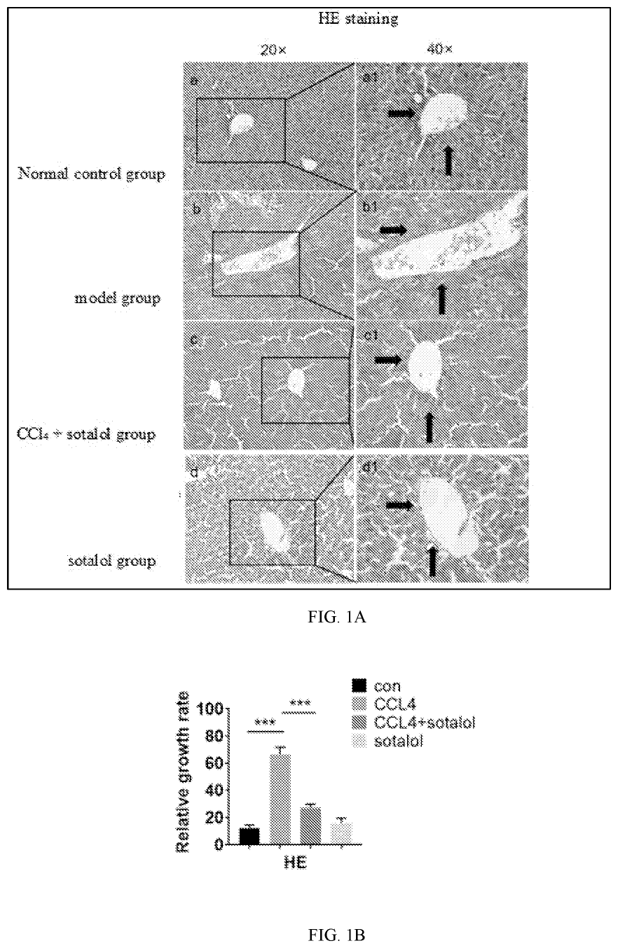

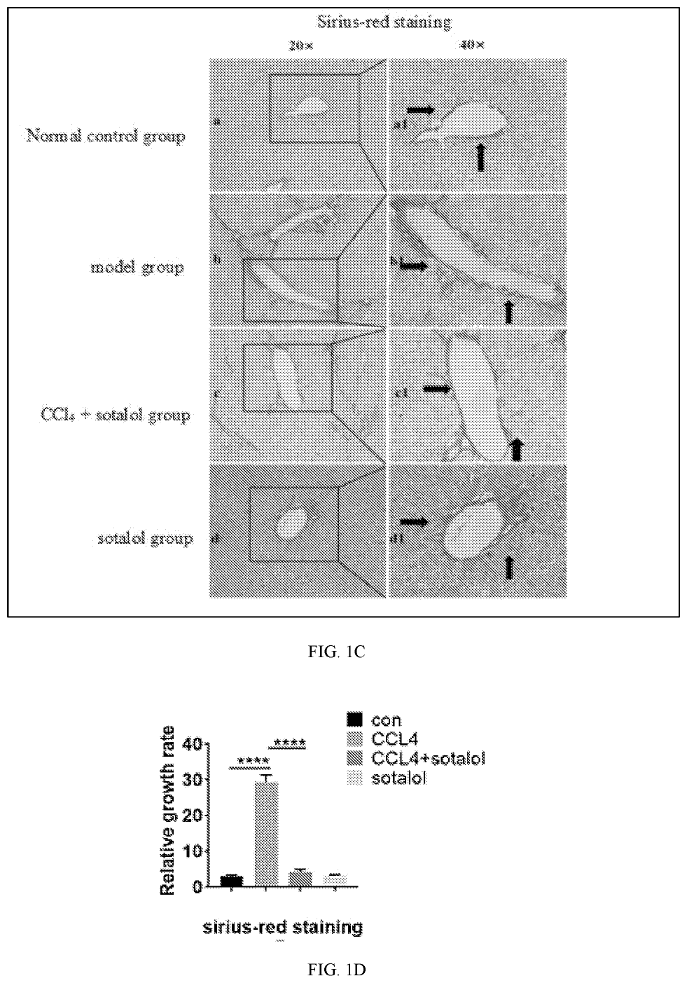

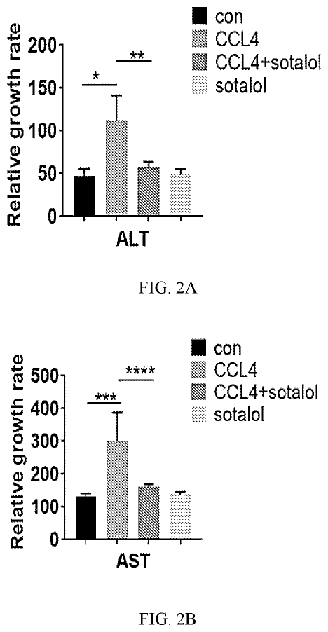

Experimental results are as follows. The results are shown in FIGS. 2A-2B, the ALT and AST levels of the eyeball blood samples obtained from the model group at the 8^{th} week are significantly increased, the staining results are shown in FIGS. 1A-1D, HE staining results show that structure of the liver tissue in the normal control group is clear, structure of hepatic lobules is normal, hepatocytes are complete and cords are clear; the liver tissue in the model group is damaged, fibrous tissue proliferates significantly; and degrees of damage of the liver tissue and fibrous tissue proliferation in the CCl₄ and sotalol group are significantly reduced. Sirius-red staining results show that the red collagen fibers in the model group are thickened and lengthened, formation of pseudo-lobule even appears in some parts, which indicates that the mouse liver fibrosis model is built successfully; and no obvious collagen fibers are observed in the sotalol group, which indicates that sotalol cannot induce liver fibrosis in mice at a concentration of 1 mM.

### Embodiment 2 Protein Expression Levels of Liver Fibrosis Indexes Alpha-Smooth Muscle Actin (α-SMA) Between Different Groups Detected by Immunohistochemistry

Experimental materials include C57 mice, α-SMA primary antibodies (also referred as anti-α-Sm-1), an immunohistochemical kit, phosphate buffered saline (PBS) solution, sodium citrate solution, and the like.

Experimental methods are as follows. The paraffin blocks prepared in the embodiment 1 are taken and performed immunohistochemistry according to the procedures after slicing into slices. The slices are soaked in 100%, 95%, 80%, and 75% ethanol for 5 minutes sequentially in that order, then rinsed them with the PBS solution, catalase is removed by using hydrogen peroxide, and antigens are repaired by sodium citrate repair solution. After cooling, the treated slices are rinsed with the PBS solution, sealed at room temperature for 30 minutes, then incubated overnight at 4° C. with primary antibodies, and incubated at room temperature with secondary antibodies for 1 hour on the next day. Then, the incubated slices are stained with diaminobenzidine (DAB), counterstained with hematoxylin, differentiated with hydrochloric acid alcohol, dehydrated, sealed, and finally observed and photographed under the microscope.

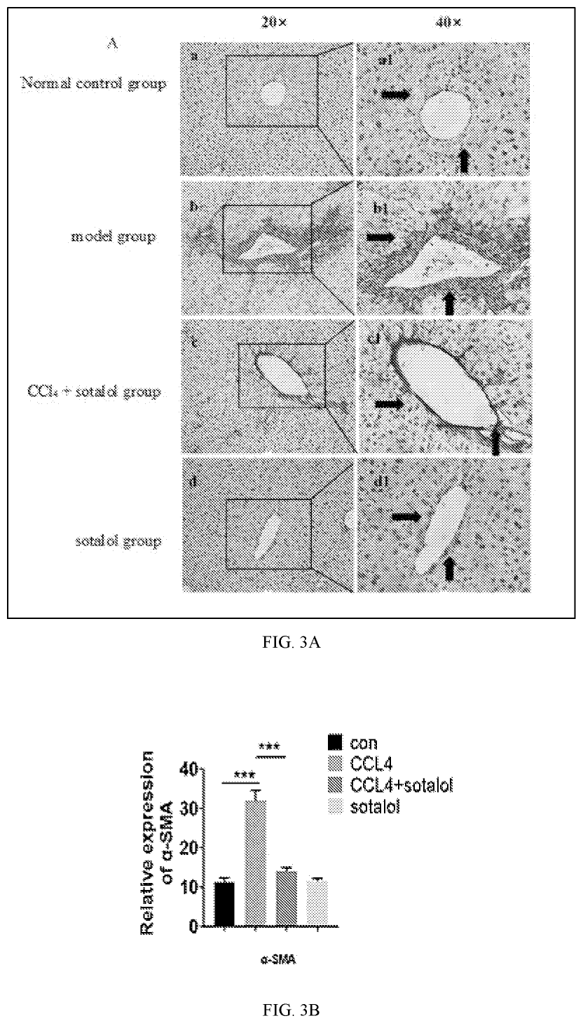

Experimental results are as follows. The results are shown in FIGS. 3A-3B, the sotalol can down-regulate expression of α-SMA in the liver fibrosis mice induced by CCl₄.

### Embodiment 3 Expression of The Liver Fibrosis Indexes α-SMA, Autophagy-Related Index, Epithelial-Mesenchymal Transition (EMT) of Different Experimental Groups By Western-Blot Detection.

Experimental materials include mouse liver tissue, a tweezer, a scissor, 2 millimeters (mm) steel balls, a magnet, a cell lysis solution, a PBS solution, a bicinchoninic acid (BCA) protein quantitative kit, defatted milk powder, and primary antibodies and corresponding secondary antibodies related to α-SMA, autophagy and EMT.

Experimental method are as follows. The liver tissue in the embodiment 1 is taken about the size of a grain of rice by using the tweezer and the scissor, added to labeled 1.5 mL Eppendorf (EP) tubes, two 2 mm steel balls and 200-250 microliter (μL) cell lysis solution are added into each of the EP tubes, after the parameters are set, and the EP tubes are placed in the grinder for full grinding. Then, the steel balls are sucked out by using the magnet, and after being applied to the ice for 30 minutes, the EP tubes are centrifuged at 12000 revolutions per minute (rpm) for 30 minutes at 4° C. A standard curve is made, 1 μL protein samples, 19 μL PBS solution and 200 μL (50:1) BCA working solution are added in each well of a 96-well plate, and two replicate wells are added per well. The 96-well plate is put in a 37°° C. drying oven for 30 minutes. The optical density (OD) value at 560 nanometers (nm) is measured by using an enzyme-linked immunosorbent assay (ELISA) on the machine to calculate the concentration of the protein. The protein is boiled in boiling water for 5 minutes before samples loading, and the samples are performed with gel electrophoresis, membrane transfer and blocking sequentially, then the treated samples are exposed after incubating with the primary antibodies and the secondary antibodies.

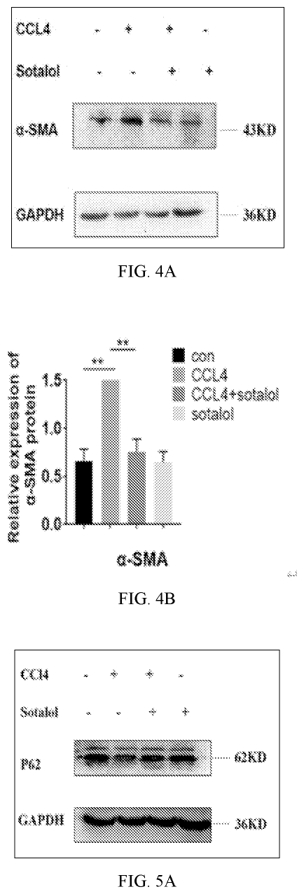

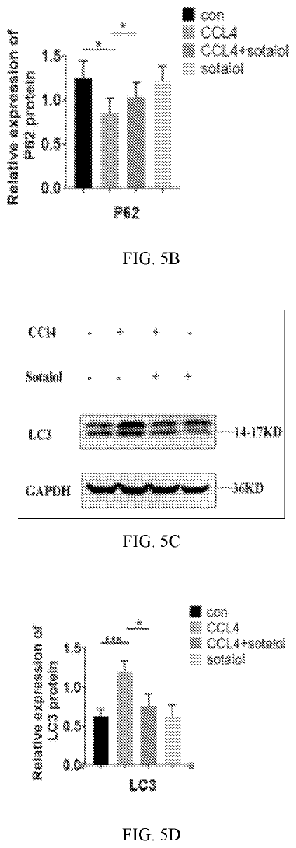

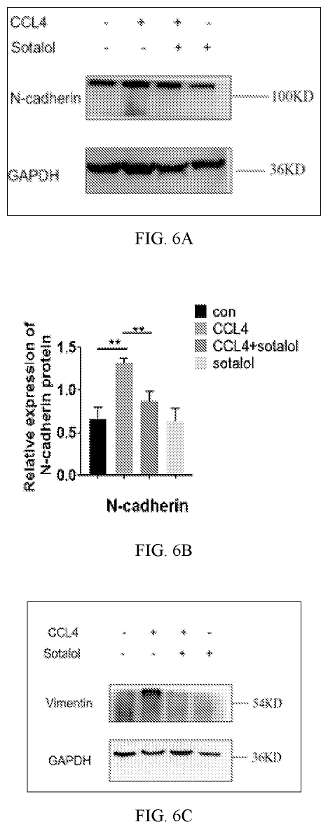

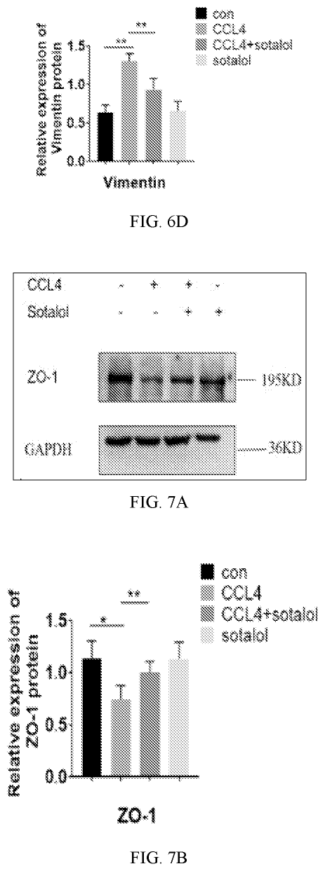

Experimental results are shown in FIGS. 4A-4B to FIGS. 7A-7B, after treatment with sotalol, expression of α-SMA and light chain 3 (LC3) in the liver tissue induced by CCl₄ are significantly reduced, expression of N-cadherin and vimentin are inhibited, and accumulation of protein 62 (p62) and zonula occludens-1 (ZO-1) are promoted.

Embodiment 4 Effects of Sotalol and Transforming Growth Factor Beta 1 (TGF-β1) Co-Culture On Proliferation of Hepatic Stellate Cells (HSCs) Detected by Cell Counting Kit-8 (CCK-8)

Experimental materials include HSC-T6 cells (also referred to as immortalized hepatic rat stellate cell line), a Dulbecco's modified Eagle's medium (DMEM) complete medium, a 96-well plate, a counting chamber (also referred to as hemocytometer), CCK-8 working solution, and the like.

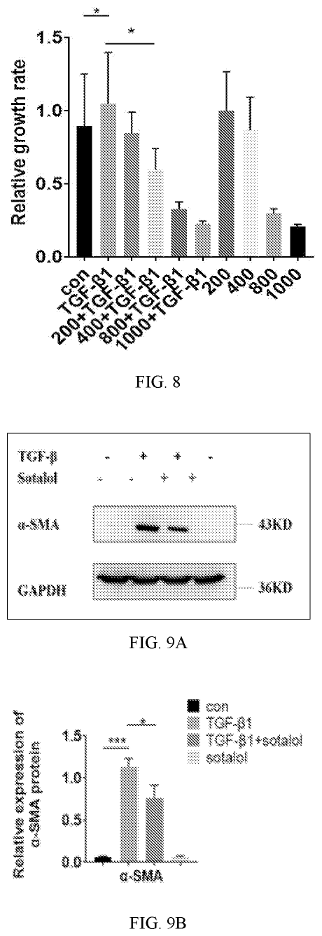

Experimental method are as follows. The HSC-T6 cells are cultured in the DMEM culture medium containing 10% fetal bovine serum, 1% double antibodies (penicillin-streptomycin solution) and 1% nonessential amino acid (NEAA) at 37° C. and 5% CO2, and the cells are digested by using trypsin for passage cultivation after the cells are converged to 80% to 90%. Next, the cells are inoculated in the plate, specifically, a density of 7×10⁶ cells per liter (cells/L) cell suspension is taken and added at 100 μL cell suspension per well to the 96-well culture plate, each group has 6 replicate wells. The experiment is divided into a normal control group (as shown in “con” in FIG. 8), a TGF-β1 group (as shown in “TGF-β1” in FIG. 8), TGF-β1 and different concentrations (200 micromoles per liter abbreviated as μM, 400 μM, 800 μM and 1000 μM) of sotalol drug groups (as shown in “200+TGF-β1”, “400+TGF-β1”, “800+TGF-β1”, “1000+TGF-β1” in FIG. 8), and sotalol drug groups (200 μM, 400 μM, 800 μM and 1,000 μM, as shown in “200”, “400”, “800”, “1,000” in FIG. 8). After cell attachment, the sotalol of corresponding concentrations are added in other groups except for the control group and the TGF-β1 group, TGF-β1 (10 nanograms per milliliter abbreviated as ng/ml) is added to the corresponding groups after 3 hours to stimulate mouse HSCs. After incubation for 48 hours, 100 μL of “10% CCK-8 and 90% medium” mixed solution is added to each well and incubated for 1 hour to 4 hours at 37° C., the wavelength is set at 450 nm, and absorbance value is read and recorded at the corresponding wavelength by using a fully-automated enzyme labeling instrument. Effects of sotalol on cell proliferation are detected by CCK-8, and an optimal time of action and an optimal drug concentration are screened.

Experimental results are shown in FIG. 8, compared with the normal value, the proliferation of HCS-T6 cells can be significantly induced after TGF-β1 stimulation, the TGF-β1-induced proliferation trend is inhibited by the co-culture of sotalol and TGF-β1.

### Embodiment 5 Expression of α-SMA, Autophagy-Related Index, and EMT in HSCs by Western-Blot Detection

Experimental materials include HSC-T6 cells, a DMEM complete medium, cell culture dishes, a cell lysis solution, a PBS solution, a BCA protein quantitative kit, defatted milk powder, primary antibodies and corresponding secondary antibodies related to α-SMA, autophagy and EMT.

Experimental method are as follows. The experiment is grouped into a normal control group, a TGF-β 1 group, a sotalol +TGF-β1 group, and a sotalol group, where the stimulation concentration of the TGF-β1 is 10 ng/mL and concentration of the sotalol is 400 μM. The HSC-T6 cells are cultured by using cell culture dishes, incubated, dosed, and added with TGF-β1according to the conditions described in the embodiment 4. After 48 hours, the incubated cells are rinsed by the precooled PBS solution for 3-4 times, and residual PBS liquid is absorbed with a filter paper, 50 μL radioimmune precipitation assay (RIPA) and 0.5 μL (100:1) phenylmethanesulfonyl fluoride (PMSF) with 100 moles per liter (mol/L) concentration are added in a 50 mL culture flask and placed on ice for 30 minutes. Then, the protein is scraped by a cell scraper from the culture flask and suctioned out into the EP tubes, and centrifuged at 12000 rpm for 30 minutes at 4° C. A standard curve is made, 1 μL protein samples, 19 μL PBS solution and 200 μL (50:1) BCA working solution are added in each well of the 96-well plate, and two replicate wells are added per well. The 96-well plate is put in a 37° C. drying oven for 30 minutes. The OD value at 560 nm is measured by using an enzyme-linked immunosorbent assay on the machine to calculate the concentration of the protein. The protein is boiled in boiling water for 5 minutes before samples loading, and the samples are performed with gel electrophoresis, membrane transfer and blocking sequentially, then the treated samples are exposed after incubating with the primary antibodies and the secondary antibodies.

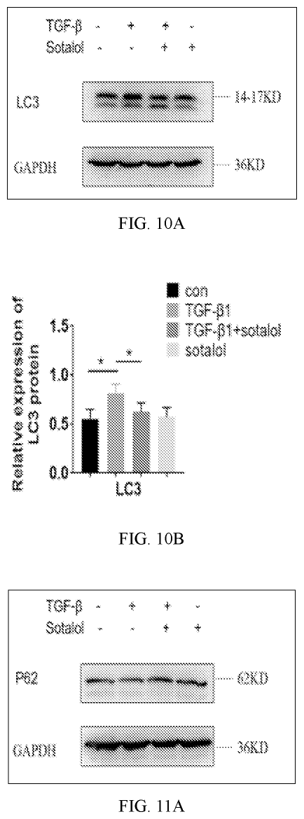

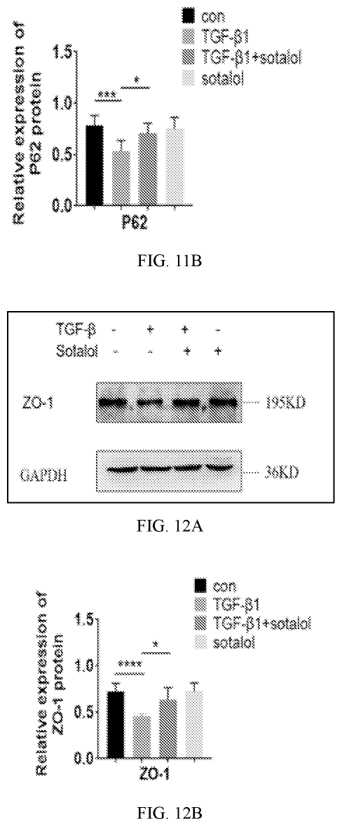

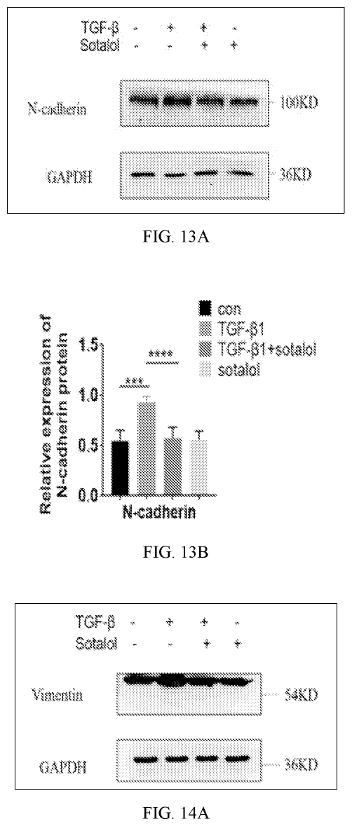

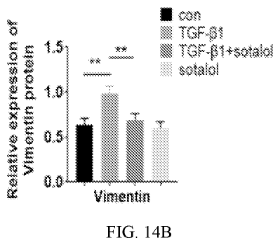

Experimental results are shown in FIGS. 9A-9B to FIGS. 14A-14B, expression of α-SMA in HSCs is significantly reduced by the sotalol. During autophagy, expression of LC3 is significantly inhibited by the sotalol, accumulation of P62 is promoted by the sotalol. During EMT, accumulation of ZO-1 can be promoted by the sotalol and expression of N-cadherin and vimentin is inhibited by the sotalol.

The above-described embodiments are merely a description of the illustrated method of the disclosure, not a limitation of the scope of the disclosure. Without departing from the spirit of the design of the disclosure, various modifications and changes made by those skilled in the art to the technical solution of the disclosure shall fall within the scope of protection determined by the claims of the disclosure.

What is claimed is:

1. An application method of potassium ion (K⁺) channel blocker, comprising preparing drugs for treating liver fibrosis by using the K⁺ channel blocker.

2. The application method according to claim 1, wherein the K⁺ channel blocker is sotalol.

3. The application method according to claim 2, wherein the sotalol is used to inhibit expression of K⁺ channels.

4. The application method according to claim 2, wherein the sotalol is used to inhibit liver fibrosis and reduce expression of a fibrosis-related protein.

5. The application method according to claim 4, wherein the fibrosis-related protein comprises alpha-smooth muscle actin (α-SMA).

6. The application method according to claim 2, wherein the sotalol is used to regulate expression of autophagy and epithelial-mesenchymal transition (EMT) molecules.

7. The application method according to claim 6, wherein the autophagy and EMT molecules comprise light chain 3 (LC3), N-cadherin and vimentin.

8. The application method according to claim 2, wherein the sotalol is used to promote accumulation of protein 62 (p62) and zonula occludens-1 (ZO-1).

9. The application method according to claim 2, wherein a concentration of the sotalol is in a range of 200 micromoles per liter (μM) to 1,000 μM.

10. The application method according to claim 9, wherein the concentration of the sotalol is 400 μM.
